(19) United States
(12) Patent Application Publication
(10) Pub. No.: US 2020/0100722 A1
(43) Pub. Date: Apr. 2, 2020

(54) Dermatological Examination

(71) Applicant: Forward Science Technologies, LLC, Stafford, TX (US)

(72) Inventors: Brian Pikkula, Sugar Land, TX (US); Robert J. Whitman, Houston, TX (US)

(73) Assignee: Forward Science Technologies, LLC, Stafford, TX (US)

(21) Appl. No.: 16/591,115

(22) Filed: Oct. 2, 2019

Related U.S. Application Data
(60) Provisional application No. 62/740,046, filed on Oct. 2, 2018.

(51) Int. Cl. A61B 5/00; A61B 90/70; A61L 2/06; A61L 2/18

(52) U.S. Cl. CPC A61B 5/444; A61B 5/0077; A61B 5/445; A61L 2202/24; A61L 2/06; A61L 2/18; A61B 90/70

(57) ABSTRACT

Provided is method of detecting a dermatological condition or abnormality associated with skin. In one embodiment, the method includes activating a dermatological examination device and directing the light emitted by the activated examination device onto human skin by orienting the activated examination device relative to the skin without touching the human with the device.

### CROSS-REFERENCE TO RELATED APPLICATIONS

This application claims the benefit of U.S. Provisional Application No. 62/740,046, filed Oct. 2, 2018, the disclosure of which is incorporated by reference herein in its entirety.

### BACKGROUND OF THE INVENTION

In dermatological screening, abnormal tissue can be distinguished from healthy tissue using different tests and/or techniques. These may include using dyes, biopsies, or cancer screening lights. However, conventional tests require the use of a disposable and/or may be limited to the particular abnormal tissue for which they are designed.

There remains a need for improved methods and devices for examining skin, e.g., human skin, to identify dermatological cancer, pre-cancer, and/or other dermatological conditions, such as allergic reactions, chemical damage, solar damage, vein disease, eczema, and/or acne, among other examples.

### BRIEF SUMMARY OF THE INVENTION

The present invention provides a method for dermatological examination. In one embodiment, the method of the invention includes activating a dermatological examination device and directing light emitted by the activated dermatological examination device onto skin, e.g., human skin, by orienting the dermatological examination device relative to the skin, to detect one or more dermatological conditions and/or abnormalities. In some embodiments, the dermatological device comprises a housing, a member coupled to the housing and comprising a light emitting diode (LED), and one or more end caps collectively comprising: a control operable to selectively activate the dermatological examination device by electrically coupling the LED to an electrical energy source; and an optical filtering component in optical alignment with light emitted by the LED.

The present invention also provides a method of detecting a dermatological condition and/or abnormality on skin, e.g., human skin, wherein the method includes activating a dermatological examination device, donning eyewear associated with the dermatological examination device, e.g., to facilitate detection of the condition or abnormality, and directing the light emitted by the examination device onto human skin by orienting the examination device relative to the human skin without touching the examination device to the human.

### BRIEF DESCRIPTION OF THE DRAWINGS

The present disclosure is best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is a perspective view of at least a portion of apparatus according to one or more aspects of the present disclosure.

FIG. 2 is a side view of the apparatus shown in FIG. 1.

FIG. 3 is an exploded view of the apparatus shown in FIGS. 1 and 2.

FIG. 4 is a schematic view of at least a portion of apparatus according to one or more aspects of the present disclosure.

FIG. 5 is a schematic view of at least a portion of a kit according to one or more aspects of the present disclosure.

FIG. 6 is a flow-chart diagram of at least a portion of a method according to one or more aspects of the present disclosure.

FIG. 7 is a schematic view of at least a portion of apparatus according to one or more aspects of the present disclosure.

FIG. 8 is a schematic view of at least a portion of apparatus according to one or more aspects of the present disclosure.

FIG. 9 is a schematic view of at least a portion of apparatus according to one or more aspects of the present disclosure.

FIG. 10 is a block diagram view of at least a portion of apparatus according to one or more aspects of the present disclosure.

FIG. 11 is a flow-chart diagram of at least a portion of a system according to one or more aspects of the present disclosure.

FIG. 12 is a block diagram of at least a portion of a processing system according to one or more aspects of the present disclosure.

FIGS. 13A-F show additional exemplary implementations of the present disclosure.

FIG. 14 shows an additional exemplary implementation of the present disclosure.

FIGS. 15A-E show additional exemplary implementations of the present disclosure.

### DETAILED DESCRIPTION OF THE INVENTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Additionally, the term “lens” as used herein may mean an optical component that diverges or converges an optical beam as well an optical component that does not converge or diverge an optical beam.

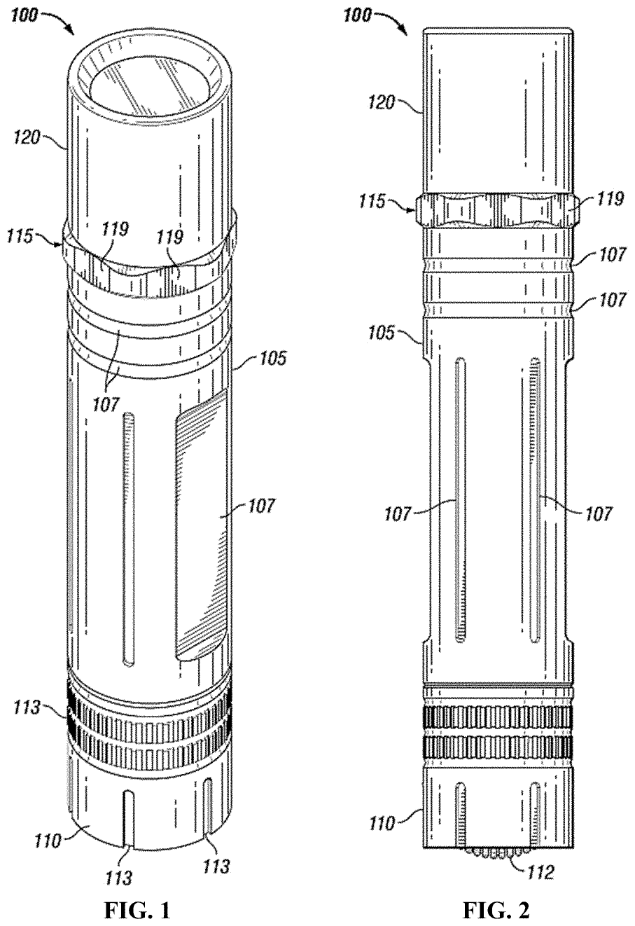

FIGS. 1-3 are perspective, front, and exploded views, respectively, of at least a portion of apparatus 100 according to one or more aspects of the present disclosure. Referring to FIGS. 1-3 collectively, the apparatus 100 may comprise a housing 105, a first end cap 110, an interstitial member 115, and a second end cap 120, all coupled end-to-end in the order listed, as shown in FIG. 1. However, the housing 105, the first end cap 110, the interstitial member 115, and the second end cap 120, a subset thereof, and/or additional components may be coupled or otherwise interconnected in a manner other than as shown in FIG. 1, and such implementations are also within the scope of the present disclosure.

The housing 105 may be or comprise a partially or substantially cylindrical tube. One or more recessed and/or protruding features 107 may be formed integrally with or otherwise coupled to the housing 105. Such features may extend axially along at least a portion of the housing 105, circumferentially around at least a portion of the housing 105, or a combination thereof, which may assist with securely gripping the apparatus 100. Opposing ends of the housing 105 may be threaded and/or otherwise configured to couple with the first end cap 110 and the interstitial member 115. For example, in the implementation depicted in FIGS. 1 and 2, the housing 105 comprises external threads to couple with the first end cap 110 and internal threads to couple with the interstitial member 115. Of course, other means for coupling the first end cap 110 and/or the interstitial member 115 with the housing 105 are also within the scope of the present disclosure. The housing 105 may comprise metal, plastic, and/or other substantially rigid materials, perhaps including materials that may be disinfected and/or sterilized various processes, such as for use in a medical office and/or other medical environments.

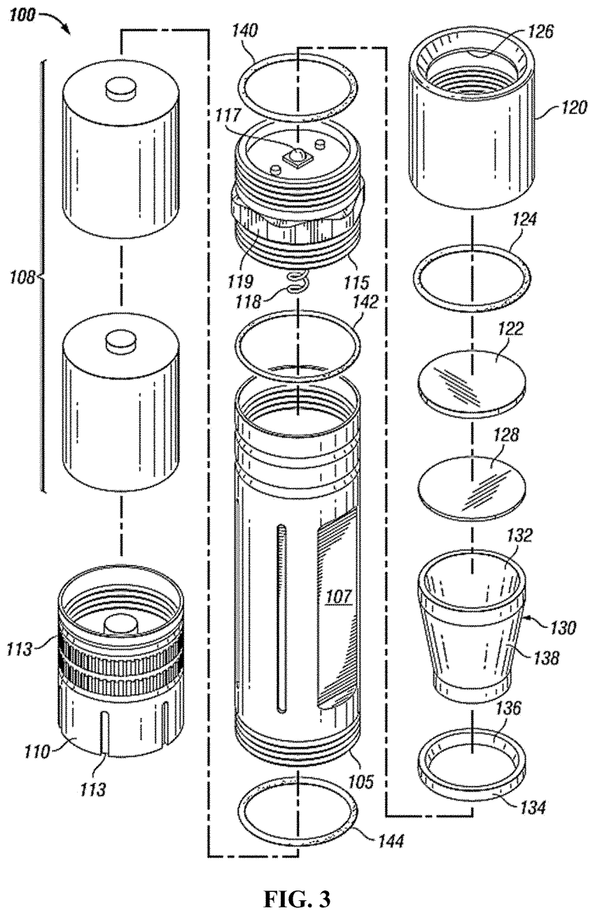

The housing 105 contains an electrical energy source 108, which in the implementation depicted in FIG. 3 comprises two batteries. The batteries may be alkaline, NiCd, NiMH, NiZn, lithium, lithium ion, silver-oxide, galvanic, electrolytic, wet cells, dry cells, and/or other types of batteries. For example, the batteries may be CR123A batteries, such as those available from ENERGIZER and DURACELL. In other implementations, the electrical energy source 108 may comprise components other than batteries, such as circuitry for connection to a facility electrical system, or a mechanically powered energy source that generates electrical power by shaking, squeezing, winding, and/or otherwise moving the housing 105 and/or another component of the apparatus 100.

The first end cap 110 may comprise a control 112 (FIG. 2) operable to electrically connect the electrical energy source 108 to other electronic components of the apparatus 100 described below. For example, the control 112 may comprise a push button control, a rotatable knob, and/or a switch, although other control types are also within the scope of the present disclosure. The first end cap 110 may also comprise one or more patterned, recessed, and/or protruding portions 113, such as may assist in removing and/or assembling the first end cap 110 to the housing 105.

The interstitial member 115 comprises a light source 117 that may be selectively energized by the electrical energy source 108 via operation of the control 112. The light source 117 may comprise one or more light emitting diodes (LEDs). For example, the light source 117 may be an LED that emits in the blue and/or violet sections of the visible spectrum, such as at a wavelength of about 450 nm or otherwise in the range of about 350 nm to about 500 nm. Intensity of the light energy emitted by the light source 117 may be about 500 mW or otherwise in the range of about 75 mW to about 2000 mW. However, other means may also or alternatively be utilized for the light source 117 within the scope of the present disclosure.

The interstitial member 115 may also comprise one or more components forming an electrical path between the electrical energy source 108 and the light source 117. For example, such an electrical path may comprise a spring and/or other member 118 extending from the interstitial member 115 to the electrical energy source 108, and/or various electronic components and/or circuitry, perhaps including one or more LED drivers, resistors, capacitors, transformers, voltage cut-off coils, low-pass filters, high-pass filters, band-stop filters, band-pass filters, integrated circuits, and/or others (not shown).

The second end cap 120 comprises an optical filtering component 122 that is in substantial optical alignment with the light source 117 and/or light emitted therefrom. The optical filtering component 122 may be or comprise a low-pass filter, a high-pass filter, a band-stop filter, and/or a band-pass filter, although other filters are also within the scope of the present disclosure. An O-ring 124 may be positioned between the optical filtering component 122 and an inside lip 126 of the second end cap 120, such as to create a seal preventing fluid from entering the apparatus via the interface between the optical filtering component 122 and the inside lip 126. The second end cap 120 may further comprise a diffuser 128 positioned to diffuse the light emitted by the light source 117 before the light is incident upon the optical filter component 122, such as to reduce the sharpness of the edges of the beam of light emitted by the light source 117. The optical filtering component 122 and/or the diffuser 128 may comprise glass, plastic, and/or other materials providing the appropriate optical characteristics.

A reflector 130 may also be positioned at least partially within the second end cap 120 in substantial optical alignment between the light source 117 and the optical filtering component 122. An internal surface 132 of the reflector 130 may be polished, painted, and/or otherwise configured to centrally focus the light energy emitted by the light source 117. The reflector 130 may comprise metal, plastic, and/or any other suitable material, and may be secured within the second end cap 120 by a retaining ring 134. For example, the retaining ring 134 may have an internal sloped surface 136 configured to cooperate with an external sloped surface 138 of the reflector 130, such that threading and/or otherwise assembling the retaining ring 134 into corresponding internal threads and/or other features of the second end cap 120 urges the sloped surfaces 136 and 138 together, thus urging the reflector 130 towards the internal lip 126 of the second end cap 120 and compressing the O-ring 124 to form the desired seal. The cooperating sloped surfaces 136 and 138 may also center the reflector 130 relative to a longitudinal axis of the second end cap 120 and/or otherwise aid in optically aligning the reflector 130 between the light source 117 and the optical filtering component 122.

At least a portion of an exterior of at least one of the housing 105, the first end cap 110, the interstitial member 115, and the second end cap 120 may have a cross-sectional shape that is not substantially circular. For example, as shown in the implementation depicted in FIGS. 1-3, a portion of the external surface of the interstitial member 115 may comprise one or more flats 119, such as may aid in preventing the apparatus 100 from rolling when resting on a substantially horizontal surface. However, one or more of the housing 105, the first end cap 110, and the second end cap 120 may alternatively or additionally comprise one or more such features. In addition to the flats 119 shown in FIGS. 1-3, or as an alternative thereto, the cross-sectional shape of the housing 105, the first end cap 110, the interstitial member 115, and/or the second end cap 120 may have at least a portion that is square, rectangular, elliptical, and/or otherwise substantially non-circular. Such features may be integrally formed with one or more corresponding components of the apparatus 100, and/or may be discrete members that are coupled to one or more of such components.

At least a portion of an exterior surface of one or more of the housing 105, the first end cap 110, the interstitial member 115, and the second end cap 120 may also comprise a material that provides substantial gripping force, such as rubber and/or other pliable materials. Such gripping features may be the same as the anti-roll features described above.

As described above, the interface between the optical filtering component 122 and the internal lip 126 of the second end cap 120 may be sealed and/or otherwise made waterproof by compression of the interposing O-ring 124. The interconnection of the other components of the apparatus 100 may also comprise similar sealing and/or other features. For example, the interface between the second end cap 120 and the interstitial member 115 may be sealed and/or otherwise made waterproof by compression of a similar O-ring 140 and/or other means. Similarly, the interface between the interstitial member 115 and the housing 105 may be sealed and/or otherwise made waterproof by compression of a similar O-ring 142 and/or other means. The interface between the housing 105 and the first end cap 110 may also be sealed and/or otherwise made waterproof by compression of a similar O-ring 144 and/or other means. The O-rings 124, 140, 142, and 144 and/or additional or alternative sealing means may seal and/or otherwise make the apparatus 100 waterproof such that, once the apparatus 100 is assembled, no fluids may enter any internal cavity of the apparatus 100.

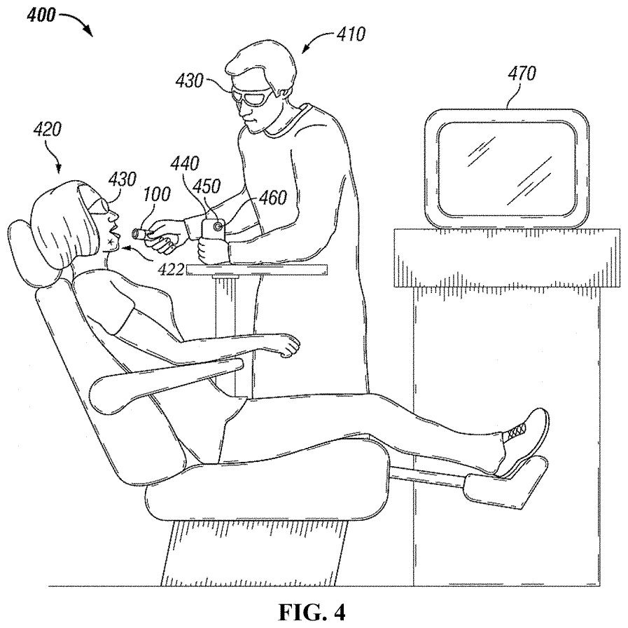

Having described various details and alternatives with respect to the apparatus 100 shown in FIGS. 1-3, operation of the apparatus 100 will now be described with reference to FIG. 4, which is a schematic view of medical personnel 410 utilizing the apparatus 100 to perform dermatological examination of a patient 420, such as to identify dermatological cancer, pre-cancer, and/or other dermatological conditions such as allergic reactions, chemical damage, solar damage, vein disease, eczema, acne, and the like. The apparatus 100 may be made commercially available as part of a kit that also contains one or more eyewear devices 430 to be worn by the medical personnel 410 and, perhaps, the patient 420 during the examination procedure.

The eyewear devices 430 may comprise an optical filtering component to be utilized in conjunction with the optical filtering component 122 shown in FIGS. 1-3. For example, the lenses of the eyewear device(s) 430 may comprise a low-pass filter, and the optical filtering component 122 of the apparatus 100 may comprise a high-pass filter. Alternatively, the lenses of the eyewear device(s) 430 may comprise a high-pass filter, and the optical filtering component of the apparatus 100 may comprise a low-pass filter. Similarly, the lenses of the eyewear device(s) 430 and the optical filtering component of the apparatus 100 may comprise different ones of a low-pass filter, a high-pass filter, a band-stop filter, a band-pass filter, and/or other types of filters. The combination of filters may be operable in conjunction with the light emitted by the apparatus 100 to detect cancerous and/or pre-cancerous regions and/or other dermatological conditions such as allergic reactions, chemical damage, solar damage, vein disease, eczema, acne, and the like of the patient 420. For example, the two filters may remove all (or substantially all) light emitted by the apparatus 100, including that which may reflect off of surfaces of the skin of the patient 420, such that only the light resulting from fluorescence of the dermatological conditions such as allergic reactions, chemical damage, solar damage, vein disease, eczema, acne, and the like remains visible to the medical personnel 410 wearing the eyewear device 430. Alternatively, the two filters and the light emitted by the apparatus 100 may work in conjunction to dim any light other than the fluorescence emanating from the dermatological conditions such as allergic reactions, chemical damage, solar damage, vein disease, eczema, acne, and the like. The patient 420 may also wear an eyewear device 430 to, for example, block at least a portion of the light emitted by the apparatus 100 from entering their eyes.

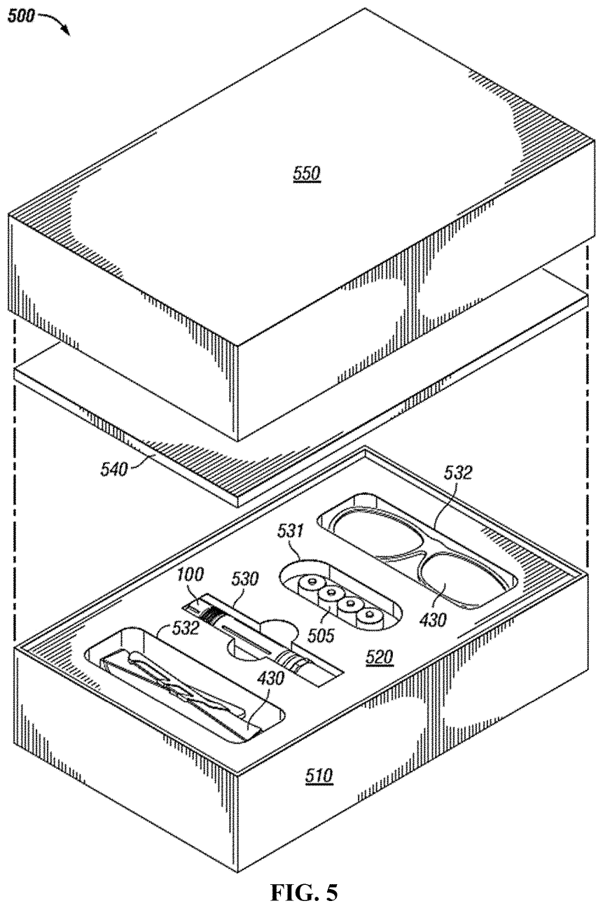

FIG. 5 is a schematic view of a kit 500 according to one or more aspects of the present disclosure. The kit 500 may comprise one or more instances of the apparatus 100 shown in FIGS. 1-4 and one or more of the eyewear devices 430 shown in FIG. 4. In the example implementation depicted in FIG. 5, the kit 500 also comprises spare batteries and/or other components 505 of the electrical energy source 108 shown in FIG. 2. The kit 500 may also or instead comprise other components described below and/or shown in subsequent figures, perhaps including apparatus shown in one or more of FIGS. 7-10.

The components of the kit 500 may be packaged in one or more boxes and/or other containers 510, perhaps within an insert 520 having recessed features 530-532 sized to receive corresponding components of the kit 500, such as may aid in protecting the components of the kit from mechanical shocks and/or other forces that may be experienced during handling, shipping, and/or otherwise. For example, the insert 520 may comprise foam and/or other shock-absorbing materials, and may have a first feature 530 sized to receive the apparatus 100 in a manner substantially preventing movement of the apparatus 100 within the container 510. Similarly, a second feature 531 may be sized to receive a predetermined number of batteries (four being shown in FIG. 5), and third features 532 may each be sized to receive one of the eyewear devices 430. Each of the second and third features 531 and 532 may also substantially prevent movement of the batteries and eyewear devices within the container 510. The kit 500 may also comprise an additional insert 540 covering the components of the kit 500 before a cover 550 is assembled to the container 510.

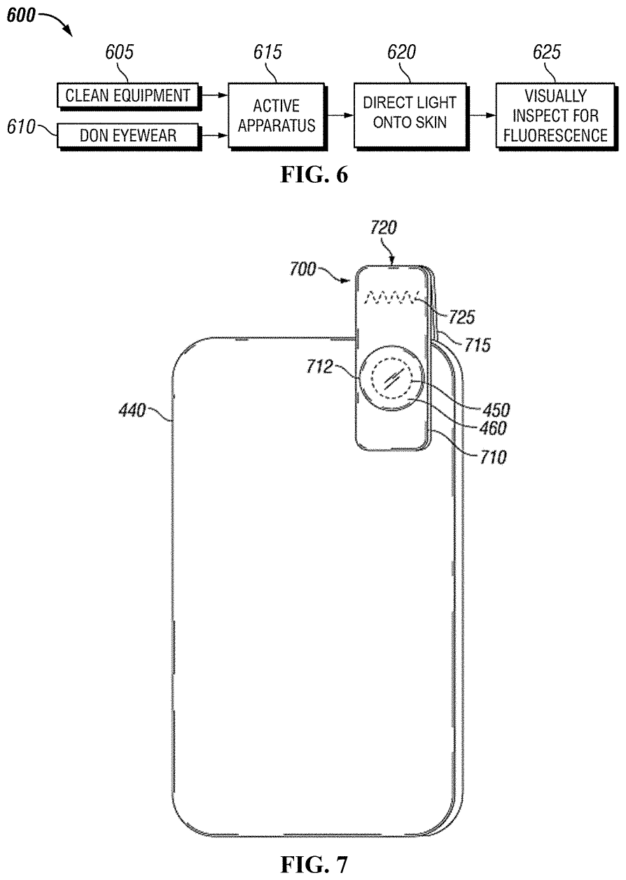

FIG. 6 is a flow-chart diagram of at least a portion of a method 600 according to one or more aspects of the present disclosure. The method 600 may utilize at least a portion of the apparatus described above, such as the apparatus 100 shown in FIGS. 1-5 and/or the eyewear devices 430 shown in FIGS. 4 and 5. The method 600 may also utilize other components described below and/or shown in subsequent figures, perhaps including apparatus shown in one or more of FIGS. 7-10.

The method 600 may comprise cleaning (605) one or more pieces of equipment to be utilized during the method 600. For example, this may entail cleaning the apparatus 100 shown in FIGS. 1-5 and/or the eyewear devices 430 shown in FIGS. 4 and 5. Such cleaning may comprise disinfecting and/or sterilizing the equipment. For example, the apparatus 100 shown in FIGS. 1-5 and/or the eyewear devices 430 shown in FIGS. 4 and 5 may be submersed in a sanitizing solution, such as CAVICIDE (available from UNIMED), MAXICIDE (available from HENRY SCHEIN, INC.), and/or isopropyl alcohol, although other disinfecting and/or sanitizing products may also or alternatively be utilized. Disinfecting and/or sterilizing the equipment may also or alternatively comprise autoclaving and/or thoroughly wiping the equipment with a sanitizing fabric, paper, and/or other wipe.

The method 600 may also comprise donning (610) an eyewear device, such as the eyewear device 430 shown in FIGS. 4 and 5, e.g., to facilitate detection of the condition or abnormality by an observer, e.g., a medical professional or the patient. This may entail one or both of the medical personnel and the patient donning the eyewear.

The light-emitting apparatus 100 shown in FIGS. 1-5 may then be activated (615). As described above, such activation may be via operation of a control of the apparatus, such as a push-button, rotating knob, switch, and/or other control. After activating the apparatus, the medical personnel may then direct (620) light emitted by the activated apparatus onto the skin of the patient. For example, the medical personnel may position the light emitting apparatus proximate a suspected abnormal portion 422 of the patient's skin and orient the apparatus such that all or a substantial portion of the emitted light impinges on the suspected abnormal portion 422. Thereafter, the medical personnel may visually inspect (625) for fluorescence emitted by cancerous areas, pre-cancerous areas, and/or other conditions such as allergic reactions, chemical damage, solar damage, vein disease, eczema, acne, and the like of the patient's skin by looking through the eyewear.

The order of the above-described portions of the method 600 may vary within the scope of the present disclosure. For example, the equipment cleaning may be performed prior to activating the light-emitting apparatus and/or after directing the emitted light onto the patient's skin. It is also noteworthy that the method 600 does not include utilizing any type of disposable that is discarded after each iteration of the method. For example, the method 600 does not include assembling a rigid or flexible plastic sleeve over the light-emitting apparatus, and thus does not include disposing of such sleeves.

Returning to the example implementation of the screening system 400 shown in FIG. 4, the medical personnel 410 may utilize a camera 450 of a mobile communications device 440 to take images of the skin of the patient 420. An optical component 460 may be operable to spectrally filter images taken by the camera 450 of the mobile communications device 440. The optical component 460 may be or comprise a high-pass, a low-pass filter, a band-stop filter, and/or a band-pass filter, although other types of light filters are also within the scope of the present disclosure. The optical component 460 may remove all (or substantially all) light emitted by the illumination source 100, including that which may reflect off of the skin of the patient 420, such that only a fluorescent signal from the tissue may remain visible to the camera 440. Thus, the optical component 460 may be at least functionally similar to the eyewear 430 donned by the medical personnel 410.

The optical component 460 may be made commercially available as part of a kit that also contains the illumination source 100, such as the kit 500 shown in FIG. 5. The kit may also comprise one or more eyewear devices 430 to be worn by the medical personnel 410 and/or the patient 420 during the dermatological screening procedure. The eyewear devices 430 may be utilized in conjunction with the illumination source 100.

The system 400 may also comprise a computer 470. The computer 470 may be operable to receive spectrally filtered image data from the mobile communications device 440 and/or other computing devices, such as those shown in subsequent figures and described below. The computer 470 may also be operable to store the image data, and perhaps to transmit the image data, such as to another mobile communications device, computer, and/or other computing device.

The mobile communications device 440 is operable to obtain, store, and transmit image data obtained utilizing the camera 450. For example, the mobile communications device 440 may be operable to transmit the image data to at least one of another mobile communications device, the computer 470, and a database. Such database may be embodied or implemented in the computer 470 or another computing device. The database, or a computing device comprising the database, may be operable to be accessed by the mobile communications device 440, another mobile communications device (such as may be remotely located), the computer 470, another computer (such as may be remotely located), and/or another database (such as may be remotely located). The database or a computing device comprising the database may also or instead be operable to transmit at least a portion of the image data to the mobile communications device 440, another mobile communications device (such as may be remotely located), the computer 470, another computer (such as may be remotely located), and/or another database (such as may be remotely located). Communication between the mobile communications device 440, another mobile communications device (such as may be remotely located), the computer 470, another computer (such as may be remotely located), the database comprising the image data received from the mobile communications device 440, and/or another database (such as may be remotely located) may be via one or more of the Internet, a cellular network, a satellite communications network, a local area network, a wireless network, a Wi-Fi connection, other wireless connections (e.g., BLUETOOTH), an Ethernet connection, a digital subscriber line (“DSL”), a telephone line, coaxial cable, and/or other means.

The image data in the database may be anonymous with respect to identification of any patient corresponding to any portion of the image data. The image data may further comprise pathology information associated with dermatological conditions such as allergic reactions, chemical damage, solar damage, vein disease, eczema, acne, and the like captured in the image data.

FIG. 7 is a schematic view of at least a portion of apparatus according to one or more aspects of the present disclosure. FIG. 7 depicts an apparatus 700 comprising the optical component 460 and is operable to hold the optical component 460 in alignment with the camera 450 of the mobile communications device 440. The apparatus 700 may comprise a filter structure 710, a contact structure 715, and an attachment structure 720. The filter structure 710 may comprise an opening 712, and the optical component 460 may be affixed within and/or otherwise held in the opening 712.

At least a portion of one or more of the filter structure 710, the contact structure 715, and the attachment structure 720 is operable to contact the mobile communications device 440. That is, the attachment structure 720 connects the filter structure 710 with the contact structure 715 in a manner operable to attach the apparatus 700 to the mobile communications device 440 and align the optical component 460 with the camera 450. For example, the attachment structure 720 may be or comprise a spring 725 operable to bias the filter structure 710 and the contact structure 715 into engagement with opposing surfaces (e.g., front and back surfaces) of the mobile communications device 460, such as by an elastic clamping and/or other type of force. However, although other arrangements are also within the scope of the present disclosure.

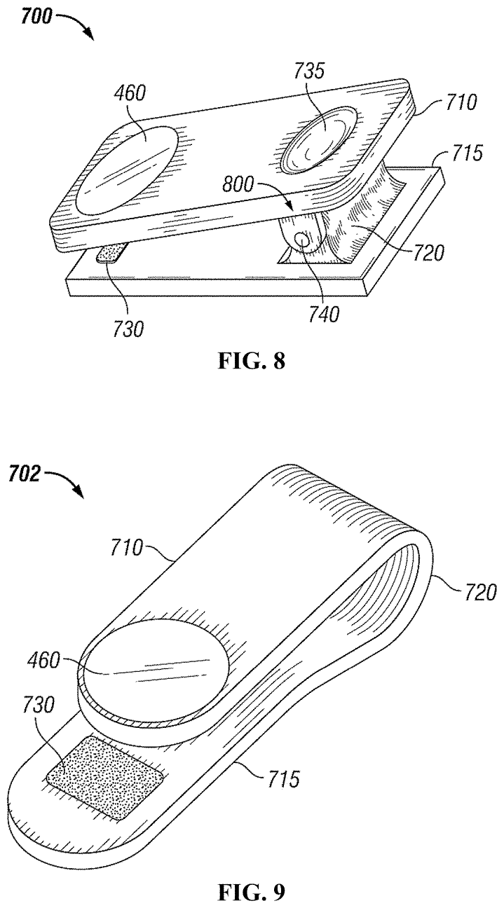

FIG. 8 is a perspective view of the apparatus 700 shown in FIG. 7 demonstrating additional, perhaps optional features that may or may not also be depicted in FIG. 7. For example, at least a portion of the filter structure 710 and/or the contact structure 715 may comprise a gripping material 730 that provides substantial gripping force, such as rubber and/or other pliable materials, which may aid in increasing friction between the apparatus 700 and the mobile communications device 440 and aligning the optical component 460 with the camera 450.

The filter structure 710 and/or the contact structure 715 may also comprise one or more finger grooves, recesses, and/or other features 735 that may aid in gripping. The attachment structure 720 may also comprise a pivot 740 and/or otherwise be operable to allow leverage and/or a pinching force to be applied and temporarily separate ends of the filter structure 710 and the contact structure 715, thus allowing insertion of the mobile communications device 440 and alignment of the optical component 460 with the camera 450.

FIG. 9 is a perspective view of another implementation of the apparatus 700 shown in FIGS. 7 and 8, designated herein by reference number 702. The apparatus 702 comprises a substantially continuous length of tempered metal and/or other elastic material that has been folded back on itself, thus forming the filter structure 710, the contact structure 715, and the attachment structure 720. The filter structure 710 may be a substantially planar length of the substantially continuous length of tempered metal and/or other elastic material, and comprises the optical component 460. The filter structure 710 may also comprise a portion of the gripping material (as described above) on an interior surface (not shown, but facing the contact structure 715) and/or other features that may be operable to aid in gripping the mobile communications device 440. The contact structure 715 may include one or more arcuate portions of the substantially continuous length of tempered metal and/or other elastic material, collectively extending in a general direction that may be substantially parallel to the filter structure 710, whether before or after assembly to the mobile communications device 440. The contact structure 715 may also comprise a portion of the gripping material 730 described above. The attachment structure 720 may be a curved portion of the substantially continuous length of tempered metal and/or other elastic material, such as may be operable to urge the filter structure 710 and the contact structure 715 towards each other, perhaps in a manner similar to the operation and/or effect of the spring 725 and/or pivot 740 described above.

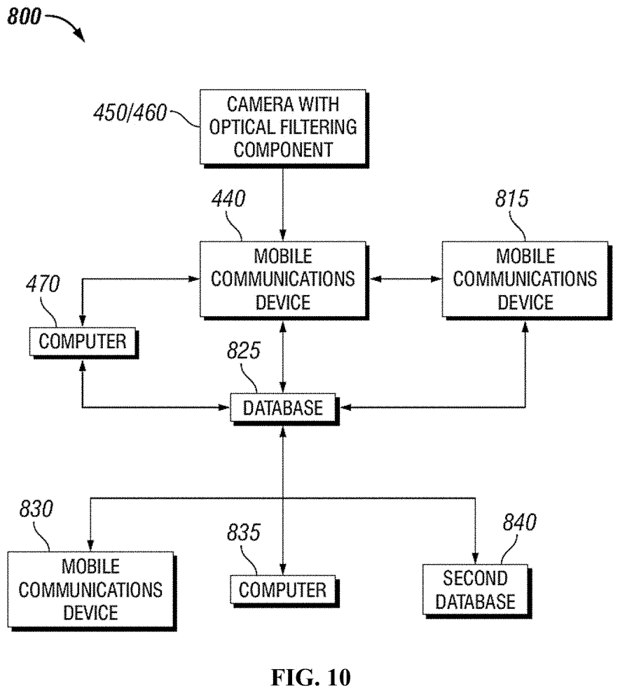

FIG. 10 is a block diagram of at least a portion of a system 800 according to one or more aspects of the present disclosure. FIG. 10 depicts an example implementation of the flow of image data spectrally filtered by the optical component 460 and captured by the camera 450 of the mobile communications device 440. Thus, the system 800 may comprise or otherwise operate in conjunction with the mobile communications device 440.

Referring to FIGS. 4 and 10, collectively, image data may be comprised of one or more images of the skin of a patient 420 taken using the camera 450 with the optical component 460. The image data may thus be received and/or stored by the mobile communications device 440. The image data may further comprise or be associated with pathology information, such as visual and/or textual information that may be associated with the dermatological images and/or conditions such as allergic reactions, chemical damage, solar damage, vein disease, eczema, acne, and the like therein. The image data may also comprise or be associated with a patient 420 corresponding to the images, such as visual and/or textual information associated with the patient 420, such as a photo of the patient's face and text identifying the patient 420. However, the image data may also be managed in a manner intending to preserve the anonymity of the patient 420.

The mobile communications device 440 may be operable to transmit image data to the computer 470, another mobile communications device 815, and/or a database 825. The computer 470 may be operable to receive, store, and transmit image data. The database 825 may be, as described above, operable to receive, store, allow access to, and transmit image data. The database 825 may receive image data from the mobile communications device 440, and may otherwise be operable to be accessed by at least one of the mobile communications device 440, the computer 470, and another database 840. The database 825 may also or instead receive image data from the computer 470 after the computer 470 has received image data from the mobile communication device 440. The database 825 may also be operable to transmit image data to at least one of the mobile communications device 440, the computer 470, and the additional database 840.

The database 825 may also be protected using one or more usernames and one or more passwords, such as where each password may grant access to the database 825 for a corresponding username. A user may use such username and password to access the database 825 and may access image data for a patient 420 stored in the database 825. Medical personnel 410 may access image data regarding a specific patient 420 via the database 825.

One or more components of the system 800 may further comprise a mobile communications program operable to allow the mobile communications device 440 to transmit, access, and receive image data utilizing the database 520. Such program may be stored in a memory and/or another non-transitory, computer-readable medium, and may include computer-readable instructions executable for capturing the image data with the mobile communications device 440, storing the image data in a memory of the mobile communications device 440, and transmitting image data from the mobile communications device 440, such as to the computer 470, the database 825, and/or another mobile communications device 815. The program may also include computer-readable instructions executable for accessing the database 825 and/or transmitting, from the database 825 to the mobile communications device 440, data not previously captured by the mobile communications device 440, perhaps including data previously captured by the additional mobile communications device 815.

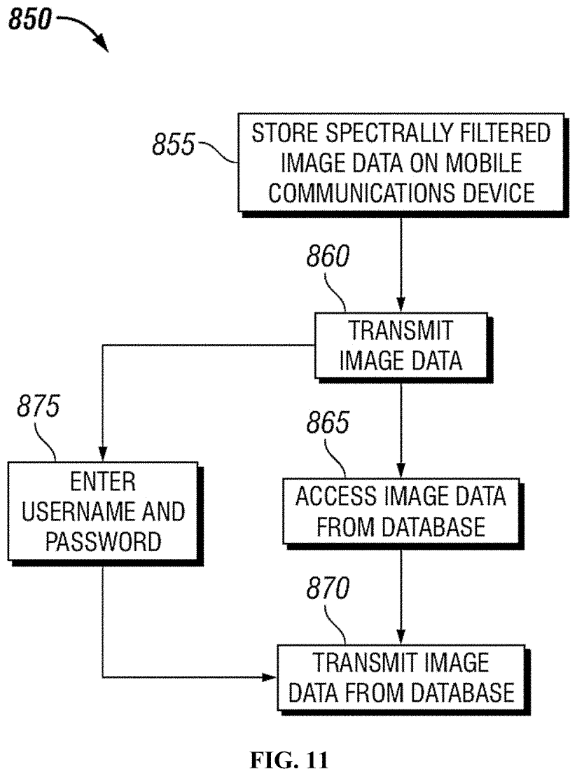

FIG. 11 is a flow-chart diagram of at least a portion of an example implementation of such a computer program 850 according to one or more aspects of the present disclosure. Referring to FIGS. 4, 10, and 11, collectively, the mobile communications program 850 includes computer-readable instructions executable to store (855) spectrally filtered image data on the mobile communications device 440 and transmit (860) image data from the mobile communications device 440, such as to the computer 470, the database 825, and/or an additional mobile communications device 815. The program 850 may also include computer-readable instructions executable to access (865) image data from the database 825, among other data locations, and transmit (870) image data from the database 825 to the mobile communications device 440. The program 850 may also or instead comprise computer-readable instructions executable to allow a username and/or password to be entered (875) prior to allowing access (865) to and/or transmission (870) of image data from the database 825.

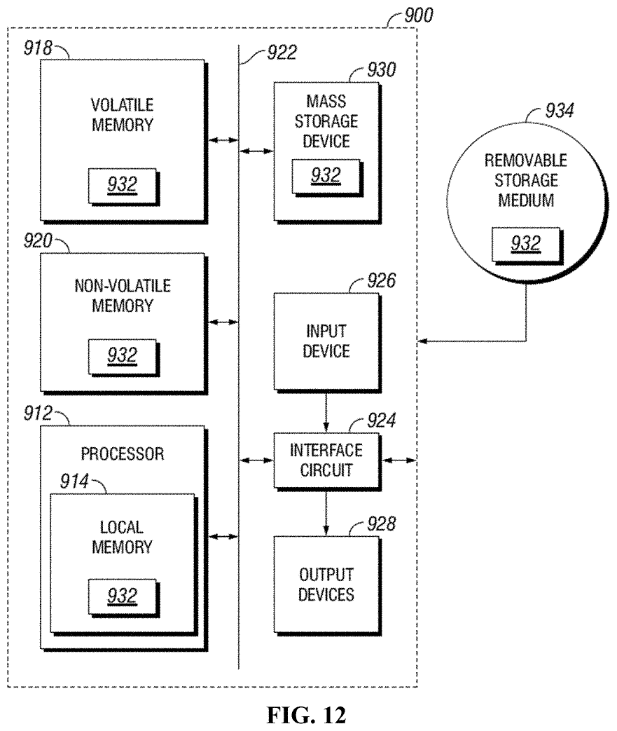

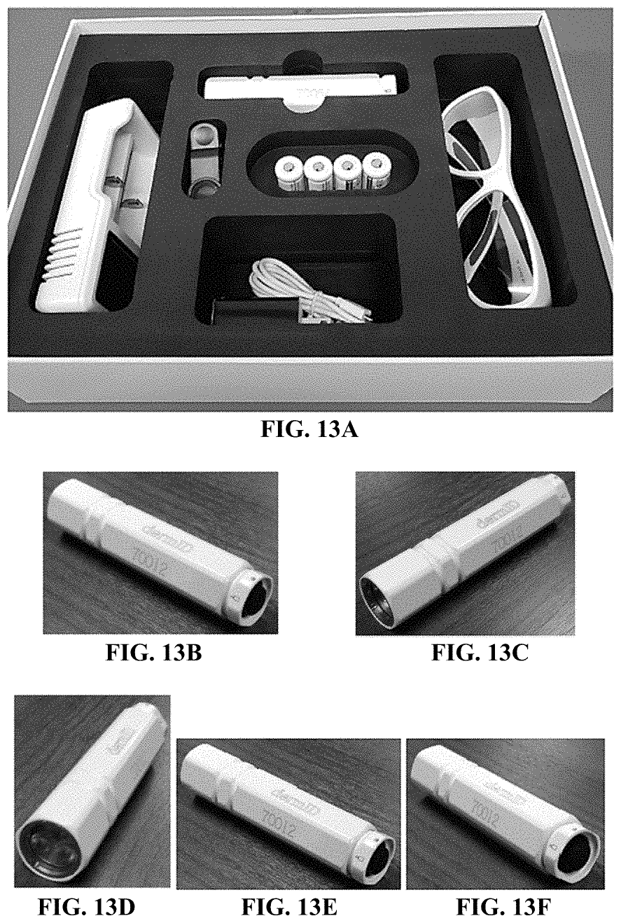

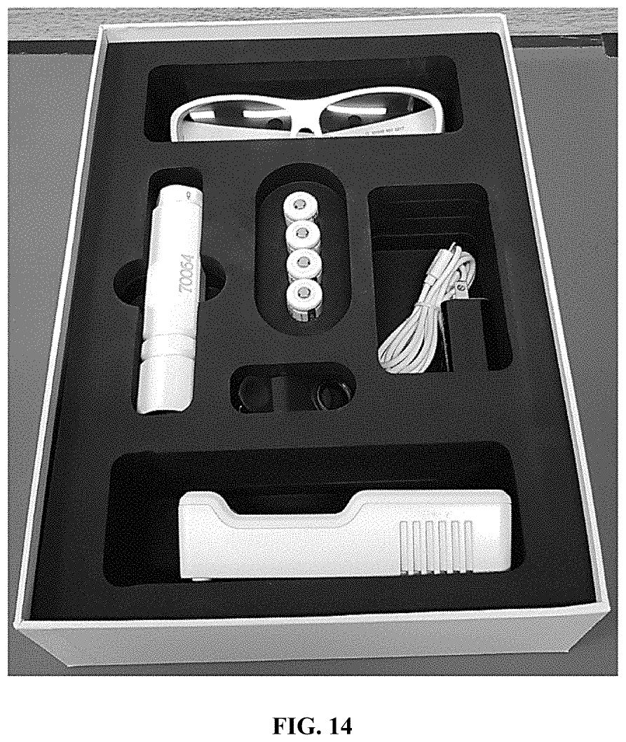

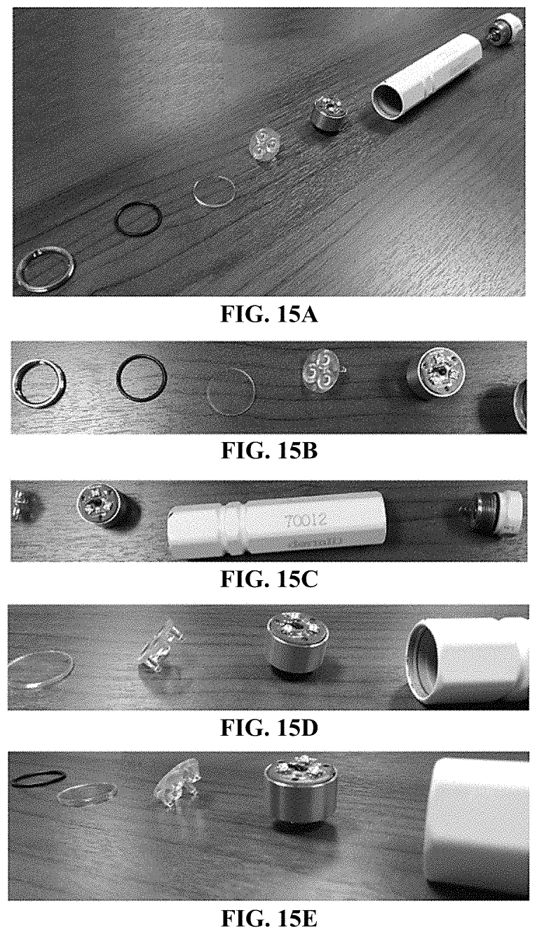

FIG. 12 is a schematic view of at least a portion of apparatus according to one or more aspects of the present disclosure. The apparatus comprises a processing system 900 that may execute example machine-readable instructions used to implement one or more of the methods and/or processes described herein, and/or to implement a portion of apparatus described herein. The processing system 900 may be or comprise, for example, one or more processors, controllers, special-purpose computing devices, servers, personal computers, personal digital assistant (“PDA”) devices, smartphones, internet appliances, and/or other types of computing devices. The processing system 900 may be implemented within one or more of the mobile communications devices shown in one or more of FIGS. 4, 7, and 10, one or more of the computers shown in one or more of FIGS. 4 and 10, and/or one or more of the databases described above with respect to FIG. 4 and/or shown in FIG. 10, perhaps including a combination thereof. Components of the processing system 900 may be utilized in the execution of the method 600 shown in FIG. 6, the method 850 shown in FIG. 11, and/or other methods and/or processes within the scope of the present disclosure.

The processing system 900 comprises a processor 912 such as, for example, a general-purpose programmable processor. The processor 912 comprises a local memory 914, and executes coded instructions 932 present in the local memory 914 and/or in another memory device. The processor 912 may execute, among other things, machine-readable instructions or programs to implement the methods and/or processes described herein. The programs stored in the local memory 914 may include program instructions or computer program code that, when executed by an associated processor, enable apparatus described herein and/or otherwise to perform tasks as described herein and/or otherwise. The processor 912 may be, comprise, or be implemented by one or more processors of various types suitable to the local application environment, and may include one or more of general-purpose computers, special purpose computers, microprocessors, digital signal processors (“DSPs”), field-programmable gate arrays (“FPGAs”), application-specific integrated circuits (“ASICs”), and processors based on a multi-core processor architecture, as non-limiting examples. Of course, other processors from other families are also appropriate.

The processor 912 is in communication with a main memory including a volatile memory 918 and a non-volatile memory 920 via a bus 922. The volatile memory 918 may be, comprise, or be implemented by random access memory (RAM), static random access memory (SRAM), synchronous dynamic random access memory (SDRAM), dynamic random access memory (DRAM), RAMBUS dynamic random access memory (RDRAM) and/or other types of random access memory devices. The non-volatile memory 920 may be, comprise, or be implemented by read only memory, flash memory and/or other types of memory devices. One or more memory controllers (not shown) may control access to the volatile memory 918 and/or the non-volatile memory 920.

The processing system 900 also comprises an interface circuit 924. The interface circuit 924 may be, comprise, or be implemented by various types of standard interfaces, such as an Ethernet interface, a universal serial bus (USB), a third generation input/output (3GIO) interface, a wireless interface, and/or a cellular interface, among others. The interface circuit 924 may also comprise a graphics driver card. The interface circuit 924 may also comprise a communication device such as a modem or network interface card to facilitate exchange of data with external computers via a network (e.g., Ethernet connection, digital subscriber line (“DSL”), telephone line, coaxial cable, cellular telephone system, satellite, etc.).

One or more input devices 926 may be connected to the interface circuit 924. The input device(s) 926 permit a user to enter data and commands into the processor 912. The input device(s) 926 may be, comprise, or be implemented by, for example, a keyboard, a mouse, a touchscreen, a track-pad, a trackball, an isopoint, and/or a voice recognition system, among others.

One or more output devices 928 may be connected to the interface circuit 924. The output devices 928 may be, comprise, or be implemented by, for example, display devices (e.g., a liquid crystal display or cathode ray tube display (CRT), among others), printers, and/or speakers, among others.

The processing system 900 may also comprise one or more mass storage devices 930 for storing machine-readable instructions and data. Examples of such mass storage devices 930 comprise floppy disk drives, hard drive disks, compact disk (CD) drives, and digital versatile disk (DVD) drives, among others. The coded instructions 932 may be stored in the mass storage device 930, the volatile memory 918, the non-volatile memory 920, the local memory 914, and/or a removable storage medium 934, such as a CD or DVD. Thus, the modules of the processing system 900 may be implemented in accordance with hardware (embodied in one or more chips including an integrated circuit such as an application specific integrated circuit), or may be implemented as software or firmware for execution by a processor. In particular, in the case of firmware or software, the embodiment can be provided as a computer program product including a computer-readable medium or storage structure embodying computer program code (i.e., software or firmware) thereon for execution by the processor.

In view of the entirety of the present disclosure, including the figures, a person having ordinary skill in the art will readily recognize that the present disclosure introduces an apparatus comprising: a housing; a member coupled to the housing and comprising a light emitting diode (LED); and one or more end caps coupled to the housing and collectively comprising: a control operable to selectively electrically couple the LED to an electrical energy source; and an optical filtering component in optical alignment with light emitted by the LED; wherein at least a portion of an exterior of at least one of the housing, the member, and the one or more end caps has a cross-sectional shape that is not substantially circular. The electrical energy source may be completely contained within the housing. The electrical energy source may comprise a battery. The electrical energy source may comprise a rechargeable battery.

At least a portion of the exterior of the housing may have the cross-sectional shape that is not substantially circular. At least a portion of the exterior of the member may have the cross-sectional shape that is not substantially circular. The housing may be substantially cylindrical. The control may comprise a push-button control, a rotatable knob, and/or a switch.

The LED may be operable to emit light in the violet and/or blue wavelength spectrum.

The optical filtering component may comprise at least one of a low-pass filter and a high-pass filter.

The member may be an interstitial member coupled to a first end of the housing, and the one or more end caps may comprise: a first end cap coupled to a second end of the housing and comprising the control; and a second end cap coupled to the interstitial member opposite the housing and comprising the optical filtering component. The second end cap may comprise a reflector optically aligned between the LED and the optical filtering component and operable to focus the light emitted by the LED before the light is incident on the optical filtering component. The apparatus may further comprise a retaining ring threadedly coupled to an interior surface of the second end cap and extending around an external circumference of the reflector. The retaining ring may have a beveled inner perimeter, and the reflector may have a beveled outer perimeter operable to slidingly engage the beveled inter perimeter of the retaining ring, thereby centering the reflector within the second end cap.

At least a portion of the exterior of at least one of the housing, the member, and the one or more end caps may have one or more substantially flat portions and/or a gripping material.

The present disclosure also introduces an apparatus comprising: a housing; an electrical energy source contained within the housing; a first end cap sealingly coupled to a first end of the housing and comprising a control; an interstitial member sealingly coupled to a second end of the housing, wherein the interstitial member comprises a light emitting diode (LED), and wherein the control is operable to selectively electrically couple the electrical energy source to the LED; and a second end cap sealingly coupled to the interstitial member opposite the housing and comprising an optical filtering component in optical alignment with light emitted by the LED, wherein the optical filtering component is sealingly coupled to an end of the second end cap opposite the interstitial member.

The housing may be substantially cylindrical.

At least a portion of the exterior of at least one of the housing, the first end cap, the interstitial member, and the second end cap may have one or more substantially flat portions and/or a gripping material.

The apparatus may further comprise a sealing member coupled between the first end cap and the first end of the housing, a sealing member coupled between the interstitial member and the second end of the housing, a sealing member coupled between the second end cap and the interstitial member, and/or a sealing member coupled between the optical filtering component and the end of the second end cap.

The electrical energy source may comprise a battery, including a rechargeable battery.

The control may comprise a push-button control, a rotatable knob, and/or a switch.

The LED may be operable to emit light in the violet and/or blue wavelength spectrum.

The optical filtering component may comprise at least one of a low-pass filter and a high-pass filter.

The second end cap may comprise a reflector optically aligned between the LED and the optical filtering component and operable to focus the light emitted by the LED before the light is incident on the optical filtering component. The apparatus may further comprise a retaining ring threadedly coupled to an interior surface of the second end cap and extending around an external circumference of the reflector. The retaining ring may have a beveled inner perimeter, and the reflector may have a beveled outer perimeter operable to slidingly engage the beveled inter perimeter of the retaining ring, thereby centering the reflector within the second end cap.

The present disclosure also introduces a method comprising: (A) activating a dermatological examination device that comprises: (i) a housing; (ii) a member coupled to the housing and comprising a light emitting diode (LED); and (iii) one or more end caps collectively comprising: (a) a control operable to selectively activate the dermatological examination device by electrically coupling the LED to an electrical energy source; and (b) an optical filtering component in optical alignment with light emitted by the LED; and (B) directing light emitted by the activated dermatological examination device onto human skin by orienting the examination device relative to the human.

The electrical energy source may be completely contained within the housing. The electrical energy source may comprise a battery. The electrical energy source may comprise a rechargeable battery.

The member may be an interstitial member, and the one or more end caps may comprise: a first end cap coupled to a first end of the housing and comprising the control; and a second end cap coupled to the interstitial member opposite the housing and comprising the optical filtering component in optical alignment with light emitted by the LED.

The method may exclude using a disposable.

The method may further comprise disinfecting and/or sterilizing the dermatological examination device.

The method may further comprise disinfecting the dermatological examination device by submersing the dermatological examination device in a sanitizing liquid.

The method may further comprise sterilizing the dermatological examination device by autoclaving the dermatological examination device.

The method may further comprise disinfecting the dermatological examination device by wiping the dermatological examination device with a sanitizing wipe.

The method may further comprise donning eyewear associated with the dermatological examination device, e.g., to facilitate detection of the condition or abnormality. Donning the eyewear may be performed by a medical personnel and the human. The eyewear may comprise an optical filtering component. The optical filtering component may comprise at least one of a low-pass filter and a high-pass filter.

The control may comprise a push-button control, and activating the dermatological examination device may comprise operating the push-button control.

The present disclosure also introduces a kit comprising: an examination device comprising: a housing; a member coupled to the housing and comprising a light emitting diode (LED); and one or more end caps collectively comprising: a control operable to selectively electrically couple the LED to an electrical energy source; and an optical filtering component in optical alignment with light emitted by the LED; and eyewear associated with the examination device and comprising an optical filtering component.

The housing may completely receive the electrical energy source therein.

The kit may further comprise a plurality of spare batteries to be positioned within the examination device.

The kit may further comprise a container containing an insert, and the insert may comprise a plurality of recesses each receiving one of the examination device and the eyewear.

At least a portion of an exterior of at least one of the housing, the member, and the one or more end caps may have a cross-sectional shape that is not substantially circular.

The member may be an interstitial member, and the one or more end caps may comprise: a first end cap coupled to the housing and comprising the control; and a second end cap coupled to the interstitial member and comprising the optical filtering component. The first end cap may be sealingly coupled to a first end of the housing. The interstitial member may be sealingly coupled to a second end of the housing. The second end cap may be sealingly coupled to the interstitial member. The optical filtering component may be sealingly coupled to the second end cap.

The kit may not include a disposable discarded after each use of the examination device.

The present disclosure also introduces a method comprising examining human skin using fluorescence with excitation in the visible spectrum to visualize or enhance the appearance, e.g., on the skin, of dermatological cancer, pre-cancer, and/or other dermatological conditions, such as allergic reactions, chemical damage, solar damage, vein disease, eczema, and/or acne, among other examples.

The method may comprise using a hand-held illumination device.

The method may comprise using a table top device.

The method may comprise using a device which illumination light emanates from an arrangement where: LEDs surround a reflective surface like a mirror; visible excitation light broadcasts onto human skin; the user dons glasses for viewing emission spectra; and the user views their skin fluorescence signal using the mirror.

The present disclosure also introduces a method utilizing a device which illumination light emanates from an arrangement where: LEDs surround a reflective surface like a mirror; visible excitation light is broadcast onto human skin; the user dons glasses for viewing emission spectra; and the user views their skin fluorescence signal using the mirror.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

Exemplary Implementations Include:

Embodiment 1. A method, apparatus, system, kit, or computer program product according to one or more aspects explicitly or implicitly described in the present disclosure.

Embodiment 2. A method comprising:

activating a dermatological examination device that comprises:
a housing;
a member coupled to the housing and comprising a light emitting diode (LED); and
one or more end caps collectively comprising:
   a control operable to selectively activate the dermatological examination device by electrically coupling the LED to an electrical energy source; and
   an optical filtering component in optical alignment with light emitted by the LED; and

directing light emitted by the activated dermatological examination device onto human skin by orienting the dermatological examination device relative to the human skin.

Embodiment 3. The method of Embodiment 2 further comprising donning eyewear associated with the dermatological examination device, e.g., to facilitate detection of the condition or abnormality.

Embodiment 4. The method of Embodiment 2 further comprising capturing a digital image of the illuminated portion of the human skin.

Embodiment 5. A method comprising:

examining human skin using fluorescence with excitation in the visible spectrum to visualize or enhance the appearance, e.g., on the skin, of dermatological cancer, pre-cancer, and/or other dermatological conditions, such as allergic reactions, chemical damage, solar damage, vein disease, eczema, and/or acne, among other examples.

Embodiment 6. The method of Embodiment 5 further comprising a hand-held illumination device.

Embodiment 7. The method of Embodiment 5 further comprising a table top device.

Embodiment 8. The method of Embodiment 5 further comprising a device which illumination light emanates from an arrangement where:

LEDs surround a reflective surface like a mirror;

visible excitation light broadcasts onto human skin;

the user dons glasses for viewing emission spectra; and

the user views the skin fluorescence signal using the mirror.

Embodiment 9. A method comprising a device which illumination light emanates from an arrangement where:

LEDs surround a reflective surface like a mirror;

visible excitation light is broadcast onto human skin;

the user dons glasses for viewing emission spectra; and

the user views their skin fluorescence signal using the mirror.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of detecting a dermatological condition or abnormality associated with skin, the method comprising:
activating a dermatological examination device that comprises:
a housing;
a member coupled to the housing and comprising a light emitting diode (LED); and
one or more end caps collectively comprising:
a control operable to selectively activate the dermatological examination device by electrically coupling the LED to an electrical energy source; and
an optical filtering component in optical alignment with light emitted by the LED; and
directing light emitted by the activated dermatological examination device onto the skin by orienting the dermatological examination device relative to the human skin to visualize or enhance the appearance on the skin of the condition or abnormality.

2. The method of claim 1, further comprising donning eyewear associated with the dermatological examination device to facilitate detection of the condition or abnormality.

3. The method of claim 1, further comprising capturing a digital image of the skin.

4. The method of claim 1, further comprising examining the skin for dermatological cancer, dermatological pre-cancer, allergic reactions, chemical damage, solar damage, vein disease, eczema, acne, or combinations thereof.

5. The method of claim 1, wherein the LED is operable to emit light in the violet or blue wavelength spectrum at an intensity from about 75 mW to about 2000 mW

6. The method of claim 1, wherein the LED emits light at a wavelength in the range of about 350 nm to about 500 nm.

7. The method of claim 6, wherein the LED emits light at a wavelength of about 450 nm.

8. The method of claim 6, wherein the LED emits light at an intensity of about 500 mW.

9. The method of claim 1, wherein the dermatological examination device is a handheld device.

10. The method of claim 1, wherein the dermatological examination device is a table top device.

11. A method of detecting a dermatological condition or abnormality associated with human skin, the method comprising:
activating a dermatological examination device that comprises:
a housing;
a member coupled to the housing and comprising a light emitting diode (LED); and
one or more end caps collectively comprising:
a control operable to selectively activate the dermatological examination device by electrically coupling the LED to an electrical energy source; and
an optical filtering component in optical alignment with light emitted by the LED,
donning eyewear associated with the dermatological examination device to facilitate detection of the condition or abnormality, and
directing the light emitted by the examination device onto human skin by orienting the examination device relative to the human skin without touching the examination device to the human.

12. The method of claim 11, further comprising examining the human skin for dermatological cancer, dermatological pre-cancer, allergic reactions, chemical damage, solar damage, vein disease, eczema, acne, or combinations thereof.

13. The method of claim 11, further comprising sterilizing the examination device, disinfecting the examination device, or a combination thereof.

14. The method of claim 13, wherein the examination device is sterilized by autoclaving the examination device.

15. The method of claim 13, wherein the examination device is disinfected by submersing the examination device in a sanitizing liquid.

16. The method of claim 13, wherein the examination device is disinfected by wiping the examination device with a sanitizing wipe.

17. A kit comprising the examination device of claim 1 and an eyewear device.

18. The kit of claim 17, further comprising a container having an insert that comprises at least two recesses for the examination device and the eyewear device.

19. The kit of claim 17, further comprising one or more disposable covers.

20. The kit of claim 19, further comprising a container having an insert that comprises at least three recesses for the examination device, the eyewear device, and the one or more disposable covers.
